(19) United States
(12) Patent Application Publication
(10) Pub. No.: US 2003/0078191 A1
(43) Pub. Date: Apr. 24, 2003

(54) OrfE-ZipA interaction and uses thereof

(76) Inventor: Steven Arthur Haney, Concord, MA (US)

Correspondence Address: Craig J. Arnold, Esq., Amster, Rothstein & Ebenstein, 90 Park Avenue, New York, NY, 10016, US

(21) Appl. No.: 10/024,644

(22) Filed: Dec. 17, 2001

Related U.S. Application Data
(60) Provisional application No. 60/257,647, filed on Dec. 22, 2000.

(51) Int. Cl. A61K 38/00

(52) U.S. Cl. 514/2

(57) ABSTRACT

The present invention provides a method for identifying an agent which interacts with ZipA. Also provided are an agent identified by this method, and a complex comprising OrfE and ZipA. The present invention further provides a method for inhibiting proliferation of a bacterium. In addition, the present invention is directed to a method for treating bacterial infection in a subject infected with a bacterium.

This application claims the benefit of U.S. Provisional Application No. 60/257,647, filed Dec. 22, 2000.

### FIELD OF THE INVENTION

The present invention relates to novel protein interaction between OrfE and ZipA. This interaction has been characterized using a yeast two-hybrid assay, deletion studies, and in vitro studies of maltose-binding protein fusions tested in a precipitation assay. An understanding of OrfE-ZipA interaction, and OrfE's role in inhibiting cell division, is beneficial for the identification and design of antimicrobial agents that inhibit ZipA activity specifically, and cell proliferation in general. Such inhibitors will be particularly useful against bacteria which contain ZipA, especially Gram-negative bacteria. Accordingly, the present invention provides methods for using the OrfE-ZipA protein interaction, as disclosed herein, to screen for agents having antimicrobial activity, and to detect agents that inhibit cell proliferation.

### BACKGROUND OF THE INVENTION

Pathogens are microorganisms that have the capacity to cause disease. Bacteria and fungi account for most of the pathogens which produce infectious diseases in host organisms. A host has numerous defense mechanisms to prevent invasion by pathogens, including natural barriers, such as the gastrointestinal (GI) tract, the genitourinary (GU) tract, the respiratory tract, and the skin; nonspecific immune responses, such as cytokine production and the inflammatory response; and specific immune responses, such as antibodies. Infection will occur when a host's defense mechanisms fail to protect the host from pathogenic invasion (35).

Gram-negative bacteria, such as aerobic cocci and bacilli, frequently cause infectious diseases. In humans, such bacterial infections may be chronic and even life-threatening. Bacterial infections can manifest in many different disease states, and embrace such serious conditions as abnormalities of mental status (e.g., coma, convulsions); cardiac manifestations (e.g., failing myocardial function, tachycardia); endocrinologic dysfunctions (e.g., increased production of hormones, profound catabolism of muscle proteins); fever; hematologic manifestations (e.g., anemia, leukocytosis, thrombocytopenia); hepatic dysfunction; renal manifestations (e.g., acute renal failure, septic shock); respiratory manifestations (e.g., hyperventilation, respiratory alkalosis); and upper GI bleeding (35).

Among the Gram-negative bacilli, the Enterobacteriaceae comprise a broad range of genera, including Enterobacter, Escherichia, Klebsiella, Salmonella, and Shigella. Escherichia coli typically inhabits the GI tract of humans and other animals. If normal anatomic barriers are disrupted, the organism may spread to adjacent structures or invade the bloodstream, resulting in bacteremia. E. coli is also an opportunistic pathogen, causing infections in patients who have defects in host resistance as a result of disease (e.g., cancer, cirrhosis, diabetes) or who have received treatment with antibiotics, antineoplastic drugs, corticosteroids, or radiation. In addition, E. coli commonly produces acute infectious neonatal diarrhea, bacteremia, meningitis, and neonatal sepsis in newborns, particularly pre-term infants (35).

When E. coli organisms have colonizing, cytotoxic, enterotoxic, or invasive virulence traits, they become major causes of disease. For example, E. coli may cause gastroenteritis, or inflammation of the lining of the stomach and intestines, a condition predominantly manifested by upper-GI-tract symptoms, such as anorexia, nausea, and vomiting, along with diarrhea (with or without blood and mucus) and abdominal discomfort. Occasionally, the hemolytic-uremic syndrome (HUS) also may occur. A potentially fatal disorder, HUS is characterized by acute renal failure, changing manifestations of ischemic damage to multiple organs, fever, and thrombocytopenia (35). Enterotoxigenic and enteropathogenic E. coli are responsible for causing diarrhea in infants and traveler's diarrhea in adults. Enterohemorrhagic strains of E. coli, such as type O157:H7, also cause watery, inflammatory, or bloody diarrhea, often associated with severe abdominal cramps and HUS. Other strains of enteroaggregative E. coli are emerging as potentially important causes of persistent diarrhea in children in tropical areas and in patients with AIDS (35).

Additionally, E. coli can be a causative pathogen in Gram-negative meningitis, which frequently occurs in immunocompromised persons or after bacteremia, CNS surgery, CNS trauma, or nosocomial infections. Acute bacterial meningitis is characterized by fever, headache, stiff neck, and vomiting, often preceded by a prodromal respiratory illness or sore throat. Brain edema, which may arise, may be further aggravated by ischemic brain damage. As intracranial pressure rises, blood pressure falls (causing septic shock), and the patient may die of systemic complications or massive brain infarction. Adults may become desperately ill within 24 hours, and children even sooner (35).

Bacterial pathogens, such as E. coli, may produce infection in a host by releasing toxins that interact with adjacent or distant cells. However, other factors may assist the microorganism in its invasion and/or infection of the host's system. These include virulence factors (e.g., capsules, enzymatic activity, and mechanisms to impair antibody production); adherence ability (via fibrillae, fimbriae, pili, or adhesins); and resistance to antimicrobial agents due to selective pressures on microbial populations (e.g., changes on a microevolutionary scale, such as point mutations; and changes on a macroevolutionary scale, such as whole-scale rearrangements of large segments of DNA resulting from insertion sequences, transposons, and acquisition of foreign DNA carried by bacteriophages, plasmids, or transposable genetic elements). Inadequate doses of antimicrobial agents promote the development of resistance. Thereafter, even greatly increased doses may fail to control the infection. Resistance to antimicrobial agents may be avoided by controlling infections promptly (35).

Antimicrobial agents act on infectious bacteria and other microorganisms by inhibiting cell wall synthesis and activating enzymes that destroy the cell wall, by increasing cell membrane permeability, by interfering with protein synthesis, and by interfering with nucleic acid metabolism. Some antimicrobial agents, such as bacteriostatic drugs (e.g., azithromycin, chloramphenicol, erythromycin, and tetracyclines), slow microbial growth. Other antimicrobial agents, such as bactericidal drugs (e.g., aminoglycosides, cephalosporins, penicillins, polymyxins, and vancomycin), kill the targeted microorganism. To treat a bacterial infection, an antimicrobial agent must be toxic to the infectious bacterium, but not toxic to the patient. This selective toxicity generally is attained by ensuring that the antimicrobial agent is administered to the patient in an amount which may be tolerated by the patient (i.e., it does not deleteriously interfere with the host's biochemical system), but which is harmful or lethal to the bacterium.

Unfortunately, the development of resistance to antimicrobial agents is a problem that grows ever more widespread as strains of bacteria evolve in response to inappropriate or excessive use of such drugs. If a bacterium becomes resistant to a particular antimicrobial agent, it then becomes essential for a new antimicrobial agent to be identified. The problem of resistance to antimicrobial agents emphasizes the constant need to find replacement drugs. The ideal replacement drug would be an antimicrobial agent that operates selectively, by interfering with the physiology or biochemistry of the target bacterium without producing toxicity in the host organism.

ZipA (Z-ring Interacting Protein A) has been identified as an essential component of the cell-division machinery of Gram-negative bacteria, including E. coli, through its direct association with FtsZ (14). FtsZ is a tubulin- like protein that forms a ring (the Z-ring) around the bacterial division site, creating the septum (15, 19). To date, the precise mechanism of the Z-ring assembly, and how it affects cell-wall invagination, remains unknown. However, using two-hybrid experiments and a co-sedimentation assay, Liu et al. determined that only the C-terminus domain of ZipA (residues 176-328) is required for interaction with FtsZ (19). Although ZipA is essential in Escherichia coli, it does not appear to be present in all prokaryotes. For example, it has not been identified in the genomes of Bacillus subtilis and Mycobacterium tuberculosis, among others. Nevertheless, it is possible that ZipA, and other genes that play a conserved role in cell division, may not be recognized by automated homology searches, such as BLAST, or that errors in contig assembly have caused important regions of conserved genes to be missed.

Cryptic prophages are resident in many E. coli strains. They are defined as prophage-defective in the lytic phase of the temperate phage life cycle, although they still express certain prophage functions, such as immunity (7, 27). The cryptic prophage, Rac, is a 25-kb fragment located at min 29.5 in many E. coli K-12 strains (8). Rac carries a killing function, identified as the product of a short open-reading frame, that causes arrest of cell division in E. coli. kil is a name commonly given to genes that interfere with cell growth. Typically, the interference is associated with the inhibition of cell division and cell lysis, particularly for kil genes found in lambdoid phages and in phage Mu.

kil genes function in promoting cell lysis as a phage takes over a host. The product of the kil locus of Rac is OrfE, a small protein that is expressed only under conditions of prophage induction (8). While this protein previously had been shown to confer a cell-division defect in E. coli, its earlier characterization suggested that conferral of this defect was mediated by an interaction with FtsZ rather than ZipA (8). Prior to the present invention, the precise mechanism by which OrfE interferes with cell division in E. coli was not known, nor was the ability of OrfE to inhibit cell division in other microorganisms.

### SUMMARY OF THE INVENTION

The present invention is based on the discovery that OrfE, a product of the kil gene of the cryptic prophage Rac, interacts with ZipA. This discovery permits screening for inhibitors of ZipA activity, which will have implications for the treatment of bacterial infections.

Accordingly, the present invention provides a method for identifying an agent which interacts with ZipA, by contacting a candidate agent with ZipA, in the presence of OrfE, and then assessing the ability of the agent to inhibit OrfE-ZipA protein interaction. Also provided are an agent identified by this method, and a complex comprising OrfE and ZipA.

The present invention further provides a method for inhibiting proliferation of a bacterium, by contacting the bacterium with an amount of an agent which interacts with ZipA, as disclosed herein, effective to inhibit proliferation.

Finally, the present invention is directed to a method for treating bacterial infection in a subject infected with a bacterium, by administering to the subject an amount of an agent which interacts with ZipA, as disclosed herein, effective to treat the infection.

Additional objects of the present invention will be apparent in view of the description which follows.

### BRIEF DESCRIPTION OF THE FIGURES

FIG. 1 depicts the interaction of ZipA and FtsZ in the yeast two-hybrid system. Cultures of haploid and diploid strains containing the indicated plasmids were aliquoted into a 96-well microtiter plate and spotted onto the plates, as indicated. Plates were incubated at 30° C. for 3 days.

FIG. 2 illustrates that OrfE interacts specifically with ZipA in the yeast two-hybrid system. Plasmids encoding the indicated fusion constructs were expressed in the SHy22×SHy23 background (A), or in the SHy19×YM4271 background (B), as diploids resulting from individual matings. Cultures were spotted, as described above, onto an SD-Leu-Trp-His/+0.5 mM AT plate (A) or an SD-Ade-Trp-His/0.5 mM AT/GalRaf plate. Plates were incubated at 30° C. for 3 days.

FIG. 3 depicts phase-contrast microscopy of E. coli cells expressing OrfE. Cells were grown overnight in NZCMG media, and inoculated at 1:100 in 2×YT media. Cells were permitted to grow for 4 hours before harvesting and viewing by phase-contrast microscopy. E. coli host strain TB1, transformed with pMal-C2×(A), pMal-C2×-FtsZ(311-383) (B), and pMal-C2×-kil (C), is shown.

FIG. 4 demonstrates that OrfE interacts with ZipA in vitro. Polyclonal αZipA antibodies were used to identify ZipA that was co-purified following incubation with an amylose resin-bound maltose-binding protein (MBP) fusion, as indicated in the figure.

FIG. 5 illustrates that OrfE inhibits cell division in Bacillus subtilis. B. subtilis strain 168, transformed with pDG148 (A), pDG148-FtsZbs (B), or pDG148-kil (C), was grown overnight in BHT media, reinoculated in fresh BHT media, and grown for an additional 5 hours before fixing and viewing by phase-contrast microscopy.

FIG. 6 depicts the complete nucleotide and amino acid sequences of E. coli ZipA (SEQ. ID. NOs. 1 and 2, respectively).

### DETAILED DESCRIPTION OF THE INVENTION

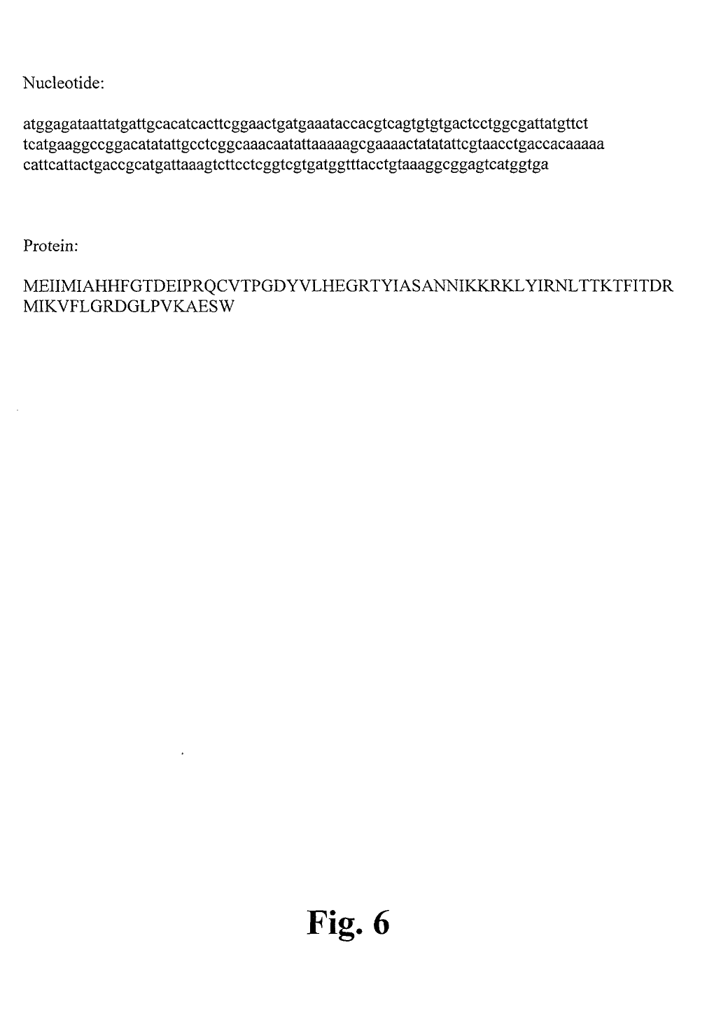

The present invention provides a method for identifying an agent which interacts with ZipA, by assessing the ability of a candidate agent to inhibit OrfE-ZipA interaction. Unless otherwise indicated, “ZipA” includes both a “ZipA peptide” and a “ZipA analogue”. A “ZipA peptide” includes at least the C-terminus domain of ZipA (including conservative substitutions thereof), from residues 176-328, up to and including a “ZipA protein” having the amino acid sequence set forth in FIG. 6 (including conservative substitutions thereof). Unless otherwise indicated, “protein” shall include a protein, protein domain, polypeptide, or peptide. A “ZipA analogue” is a functional variant of the ZipA peptide, having ZipA biological activity, that has 80% or greater (preferably, 90% or greater) amino-acid-sequence homology with the ZipA peptide. As further used herein, the term “ZipA biological activity” refers to the activity of a protein or peptide that demonstrates detectable binding with OrfE (i.e., binding of approximately two fold, or, more preferably, approximately five fold, above the background binding of a negative control), under the conditions of the assays described herein, although affinity may be different from that of ZipA.

Similarly, unless otherwise indicated, “OrfE” includes both an OrfE protein and an “OrfE analogue”. An “OrfE analogue” is a functional variant of the OrfE peptide, having OrfE biological activity, that has 80% or greater (preferably, 90% or greater) amino-acid-sequence homology with the OrfE peptide. As further used herein, the term “OrfE biological activity” refers to the activity of a protein or peptide that demonstrates detectable binding with ZipA (i.e., binding of approximately two fold, or, more preferably, approximately five fold, above the background binding of a negative control), under the conditions of the assays described herein, although affinity may be different from that of OrfE.

As used herein, “conservative substitutions” are those amino acid substitutions which are functionally equivalent to the substituted amino acid residue, either because they have similar polarity or steric arrangement, or because they belong to the same class as the substituted residue (e.g., hydrophobic, acidic, or basic). The term “conservative substitutions”, as defined herein, includes substitutions having an inconsequential effect on the ability of OrfE to interact with ZipA, particularly in respect of the use of said interaction for the identification and design of ZipA inhibitors, for molecular replacement analyses, and/or for homology modeling.

It will be obvious to the skilled practitioner that the numbering of amino acid residues in the various isoforms of ZipA, or in ZipA analogues covered by the present invention, may be different than that set forth herein, or may contain certain conservative amino acid substitutions that produce the same OrfE-binding activity as that described herein. Corresponding amino acids and conservative substitutions in other isoforms or analogues are easily identified by visually inspecting the relevant amino acid sequences, or by using commercially-available homology software programs.

The ZipA of the present invention may be obtained from bacteria, particularly Gram-negative bacteria. Preferably, the ZipA of the present invention is obtained from Escherichia coli. The OrfE of the present invention may be obtained from phages, particularly lambdoid phages. Preferably, the OrfE of the present invention is obtained from the kil gene of the cryptic prophage, Rac (8).

The present invention also contemplates the use of proteins and protein analogues generated by synthesis of polypeptides in vitro, e.g., by chemical means or in vitro translation of mRNA. For example, ZipA or OrfE may be synthesized by methods commonly known to one skilled in the art (33, 34). Examples of methods that may be employed in the synthesis of the ZipA and OrfE amino acid sequences, and analogues of these sequences, include, but are not limited to, solid-phase peptide synthesis, solution-method peptide synthesis, and synthesis using any of the commercially-available peptide synthesizers. The ZipA and OrfE amino acid sequences of the present invention may contain coupling agents and protecting groups, which are used in the synthesis of protein sequences, and which are well-known to one of skill in the art.

The method of the present invention comprises the steps of: (a) contacting a candidate agent with ZipA, in the presence of OrfE; and (b) assessing the ability of the candidate agent to inhibit OrfE-ZipA interaction. As used herein, an “agent” shall include a protein, polypeptide, peptide, nucleic acid (including DNA or RNA), antibody, Fab fragment, F(ab′)₂ fragment, molecule, compound, antibiotic, drug, and any combinations thereof. A Fab fragment is a univalent antigen-binding fragment of an antibody, which is produced by papain digestion. A F(ab′)₂ fragment is a divalent antigen-binding fragment of an antibody, which is produced by pepsin digestion. Moreover, an agent which binds to ZipA may be either natural or synthetic. For example, the agent of the present invention may be an antibody reactive with ZipA. Alternatively, the agent may be an enzyme which interacts with ZipA. As used herein, “interacts with ZipA” means the agent has affinity for, binds to, or is directed against ZipA.

The antibody of the present invention may be polyclonal or monoclonal, and may be produced by techniques well known to those of skill in the art. Polyclonal antibody, for example, may be produced by immunizing a mouse, rabbit, or rat with purified ZipA. Monoclonal antibody may then be produced by removing the spleen from the immunized mouse, and fusing the spleen cells with myeloma cells to form a hybridoma which, when grown in culture, will produce a monoclonal antibody. The antibody of the present invention also includes a humanized antibody, made in accordance with procedures known in the art.

An agent which interacts with ZipA, as disclosed herein, would have the ability to inhibit OrfE-ZipA interaction by binding to ZipA in the place of OrfE, thereby inhibiting the interaction of OrfE and ZipA. According to the method of the present invention, such an agent which interacts with ZipA may be identified using an in vitro assay (e.g., direct binding assay, competitive binding assay, etc.). For example, in a direct binding assay, the binding of a candidate agent to ZipA or a peptide fragment thereof may be measured directly.

Alternatively, in a competitive binding assay, standard methodologies may be used in order to assess the ability of a candidate agent to displace or replace OrfE in its binding to ZipA, thereby inhibiting the interaction of OrfE and ZipA. In such a competitive binding assay, the candidate agent competes with OrfE for binding to ZipA, and, once bound to ZipA, sterically hinders binding of OrfE to ZipA. A competitive binding assay represents a convenient way to assess inhibition of OrfE-ZipA interaction, since it allows the use of crude extracts containing ZipA and OrfE. A competitive binding assay may be carried out by adding ZipA, or an extract containing ZipA biological activity, to a mixture containing the candidate agent and labeled OrfE, both of which are present in the mixture in known concentrations. After incubation, the ZipA-agent complex may be separated from the unbound labeled OrfE and unlabeled candidate agent, and counted. The concentration of the candidate agent required to inhibit 50% of the binding of the labeled OrfE to ZipA (IC₅₀) then may be calculated.

The binding assay formats described herein employ labeled assay components. Labeling of OrfE or ZipA may be accomplished using one of a variety of different chemiluminescent and radioactive labels known in the art. The label of the present invention may be, for example, a nonradioactive or fluorescent marker, such as biotin, fluorescein (FITC), acridine, cholesterol, or carboxy-X-rhodamine, which can be detected using fluorescence and other imaging techniques readily known in the art. Alternatively, the label may be a radioactive marker, including, for example, a radioisotope. The radioisotope may be any isotope that emits detectable radiation, including, without limitation, ³⁵S, ³²P, ¹²⁵I, ³H, or ¹⁴C. Qualitative results then may be obtained by competitive autoradiographic-plate binding assays; alternatively, Scatchard plots may be used to generate quantitative results. The labels of the present invention may be coupled directly or indirectly to the desired component of the assay, according to methods well known in the art. The choice of label depends on a number of relevant factors, including the sensitivity required, the ease of conjugation with the compound to be labeled, stability requirements, and available instrumentation.

Both direct and competitive binding assays may be used in a variety of different configurations. In one competitive binding assay, for example, the candidate agent may compete against labeled OrfE (the labeled analyte) for a specific binding site on ZipA (the capture agent) that is bound to a solid substrate, such as a column chromatography matrix or tube. Alternatively, the candidate agent may compete for a specific binding site on labeled ZipA (the labeled analyte) against wild type OrfE or a fragment thereof (the capture agent) that is bound to a solid substrate. The capture agent is bound to the solid substrate in order to effect separation of bound labeled analyte from the unbound labeled analyte. In either type of competitive binding assay, the concentration of labeled analyte that binds the capture agent bound to the solid substrate is inversely proportional to the ability of a candidate agent to compete in the binding assay. The amount of inhibition of labeled analyte by the candidate agent depends on the binding assay conditions and on the concentrations of candidate agent, labeled analyte, and capture agent that are used.

Another competitive binding assay may be conducted in a liquid phase. In this type of assay, any of a variety of techniques known in the art may be used to separate the bound labeled analyte (which may be either OrfE or ZipA) from the unbound labeled analyte. Following such separation, the amount of bound labeled analyte may be determined. The amount of unbound labeled analyte present in the separated sample is inversely proportional to the amount of bound labeled analyte.

In the further alternative, a homogeneous binding assay may be performed, in which a separation step is not needed. In this type of binding assay, the label on the labeled analyte (which may be either OrfE or ZipA) is altered by the binding of the analyte to the capture agent. This alteration in the labeled analyte results in a decrease or increase in the signal emitted by the label, so that measurement of the label at the end of the binding assay allows for detection or quantification of the analyte.

Under specified assay conditions, a candidate agent is considered to be capable of inhibiting the binding of OrfE to ZipA in a competitive binding assay if the amount of binding of the labeled analyte to the capture agent is decreased by 50% (preferably 90%) or more. When a direct binding assay configuration is used, a candidate agent is considered to bind ZipA when the signal measured is twice the background level or higher. Furthermore, as proof of the specificity of the candidate agent identified using an OrfE competitive binding assay, binding competition also may be performed using purified ZipA in the presence of washed ribosomes.

Once the candidate agent of the present invention has been screened, and has been determined to have suitable binding affinity to ZipA (i.e., it interacts with ZipA), it may be evaluated to ascertain whether it has an effect on bacterial proliferation. In particular, the candidate agent may be assessed for its ability to act as an inhibitor to cell division or to otherwise function as an appropriate antimicrobial agent.

Accordingly, the method of the present invention further comprises the steps of: (c) contacting the candidate agent with a bacterium; and (d) determining if the agent has an effect on proliferation of the bacterium. As used herein, “proliferation” includes, without limitation, growth, multiplication, replication, and reproduction of a bacterium. Examples of bacteria with which the candidate agent may be contacted include Bacillus subtilis and a bacterium which contains ZipA. As further used herein, a bacterium which “contains ZipA” refers to any bacterium in which ZipA, or a derivative or homologue thereof, is naturally expressed or naturally occurs. Such bacteria would include Gram-negative bacteria. Preferably, the bacterium which contains ZipA is Escherichia coli.

According to the method of the present invention, a candidate agent may be contacted with a bacterium in vitro. For example, a culture of the bacterium may be incubated with a preparation containing the candidate agent. The candidate agent's effect on proliferation of the bacterium then may be assessed by any of biological assays or methods known in the art, including visual assays for turbidity of plates containing bacterial suspensions. Histological analysis is also possible.

The present invention is further directed to an agent identified by the above-described identification method. Such an agent may be useful for treating bacterial infection in a subject infected with a bacterium, according to the methods described below. The bacterial infection may be treated by administering to the subject an amount of the agent effective to treat the infection. The amount of agent required to treat the bacterial infection may be readily determined by one skilled in the art.

The present invention also provides a pharmaceutical composition comprising the agent identified by the above-described identification method and a pharmaceutically-acceptable carrier. The pharmaceutically-acceptable carrier must be “acceptable” in the sense of being compatible with the other ingredients of the composition, and not deleterious to the recipient thereof. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc, and water, among others.

Formulations of the pharmaceutical composition of the present invention may be conveniently presented in unit dosage. Furthermore, the formulations may be prepared by methods well-known in the pharmaceutical art. For example, the active compound may be brought into association with a carrier or diluent, as a suspension or solution. Optionally, one or more accessory ingredients (e.g., buffers, flavoring agents, surface active agents, and the like) also may be added. The choice of carrier will depend upon the route of administration. The pharmaceutical composition would be useful for administering to a subject an agent which interacts with ZipA, in order to treat bacterial infection, according to the methods described below. Where the pharmaceutical composition is administered to a subject to treat bacterial infection, the agent which interacts with ZipA is provided in an amount which is effective to treat the bacterial infection in the subject. This amount may be readily determined by the skilled artisan.

The present invention also provides a complex comprising OrfE and ZipA. As described above, “OrfE” includes both an OrfE protein and an “OrfE analogue”, and “ZipA” includes both a “ZipA peptide” and a “ZipA analogue”. In such an OrfE-ZipA complex, amino acid residues of an OrfE-binding site of ZipA are in direct van der Waal and/or hydrogen bond and/or salt-bridge contact with the amino acid residues of OrfE.

The complex of the present invention may comprise the full amino acid sequence of ZipA complexed with the full amino acid sequence of OrfE. In another embodiment, the complex of the present invention comprises at least the C-terminus domain of ZipA, which contains an OrfE-binding site of ZipA. As used herein, the “C-terminus domain of ZipA” means residues 176-328 of ZipA, as well as analogues thereof. Moreover, as used herein, a “binding site” refers to a region of a molecule or molecular complex that, as a result of its shape and charge potential, favorably interacts or associates with another agent—including, without limitation, a protein, polypeptide, peptide, nucleic acid (including DNA or RNA), molecule, compound, antibiotic, or drug—via various covalent and/or non-covalent binding forces. Accordingly, as contemplated by the present invention, an “OrfE-binding site of ZipA” is a binding site on ZipA that, as a result of its shape, reactivity, charge, potential, and other characteristics, favorably interacts or associates with another agent, including, without limitation, a protein (e.g., OrfE), polypeptide, peptide, nucleic acid (e.g., DNA or RNA), molecule, compound, antibiotic, or drug.

An OrfE-binding site of ZipA may include the actual site on ZipA of OrfE binding. An OrfE-binding site also may include accessory binding sites, adjacent or proximal to the actual site of OrfE binding, that nonetheless may affect ZipA or OrfE-ZipA activity upon interaction or association with a particular agent—either by direct interference with the actual site of OrfE binding, or by indirectly affecting the steric conformation or charge potential of the ZipA molecule, and thereby preventing or reducing OrfE binding to ZipA at the actual site of OrfE binding.

Identification of a binding site of a molecule or molecular complex is important because the biological activity of the molecule or molecular complex frequently results from interaction between an agent/ligand and one or more binding sites of the molecule or molecular complex. Therefore, localization of an OrfE-binding site of ZipA provides the most suitable tool for identifying inhibitors which affect the activity of ZipA or FtsZ-ZipA. Localization of an OrfE-binding site of ZipA also permits the use of various molecular design and analysis techniques for the purpose of designing and synthesizing chemical agents capable of favorably associating or interacting with an OrfE-binding site of ZipA or a ZipA analogue, wherein said chemical agents potentially act as inhibitors of ZipA or FtsZ-ZipA activity.

In view of the foregoing, the OrfE-ZipA interaction and the OrfE-ZipA complex of the present invention may be used as tools in the development of drug screens, as a target for small-molecule inhibitors that can act as antimicrobial agents, and as a basis for peptidomimetics. Such drugs, inhibitors, and peptidomimetics may be useful for treating a subject infected with a bacterium, by administering to the subject an effective amount of the drug, inhibitor, or peptidomimetic that has been designed in accordance with the method of the present invention.

The design and synthesis of an inhibitor of ZipA biological activity, based on the present invention, should be relatively simple for two reasons: (1) the OrfE-binding site of ZipA, as localized herein, is a relatively small peptide sequence (contained within residues 176-328 of ZipA); and (2) the substrate of the OrfE-binding site of ZipA, as described in the present invention, is OrfE—a small, 78-residue protein of known molecular structure. ZipA is a particularly attractive target for rational drug design because this protein, which is commonly found in Gram-negative bacteria, is not found in human cells; therefore, a ZipA or FtsZ-ZipA inhibitor would not be expected to display toxicity for human cells.

The present invention further provides a method for inhibiting proliferation of a bacterium. As described above, “proliferation” includes, without limitation, growth, multiplication, replication, and reproduction of a bacterium. The method of the present invention comprises contacting the bacterium with an amount of an agent which interacts with ZipA, as disclosed herein, that is effective to inhibit proliferation of the bacterium. Examples of bacteria which may be used in the method of the present invention include, without limitation, Bacillus subtilis and a bacterium which contains ZipA, as defined above. Such bacteria would include Gram-negative bacteria. In one embodiment of the present invention, the bacterium which contains ZipA is Escherichia coli.

According to the method of the present invention, a bacterium may be contacted, either in vitro or in vivo, with an agent which interacts with ZipA, as disclosed herein. When in vivo, contacting between the bacterium and the agent may be effected by any of the methods of administration (e.g., oral or parenteral) described below. When in vitro, a culture of the bacterium, for example, may be incubated with a preparation containing the candidate agent. The candidate agent's effect on proliferation of the bacterium then may be assessed by any of the biological assays or methods known in the art, including visual assays for turbidity of plates containing bacterial suspensions. Histological analysis is also possible.

The present invention is also directed to a method for treating bacterial infection in a subject infected with a bacterium. As used herein, a “subject” is a mammal, including, without limitation, a cow, dog, human, monkey, mouse, pig, or rat, but is preferably a human. Examples of bacterial infections which may be treated by the method of the present invention include, without limitation, infections caused by Bacillus subtilis or a bacterium which contains ZipA. As defined above, a bacterium which “contains ZipA” refers to any bacterium in which ZipA, or a derivative or homologue thereof, is naturally expressed or naturally occurs. Bacteria which contain ZipA would include Gram-negative bacteria. Preferably, the bacterium which contains ZipA is Escherichia coli.

The method of the present invention comprises administering to a subject infected with a bacterium an agent which interacts with ZipA, as disclosed herein, in an amount effective to treat the bacterial infection in the subject. As used herein, the phrase “effective to treat the bacterial infection” means effective to ameliorate or minimize the clinical impairment or symptoms resulting from infection with a bacterium. For example, where the subject is infected with E. coli, the amount of agent which is effective to treat the bacterial infection is that which can ameliorate or minimize the symptoms of the E. coli bacterial infection, including, without limitation, anorexia, nausea, and vomiting, along with diarrhea (with or without blood and mucus), and abdominal discomfort (where gastroenteritis or E. coli O157:H7 infection has occurred); acute renal failure, changing manifestations of ischemic damage to multiple organs, fever, and thrombocytopenia (where hemolytic-uremic syndrome (HUS) has occurred); and fever, headache, stiff neck, and vomiting (where acute bacterial meningitis has occurred). The amount of agent effective to treat an infection in a subject infected with a bacterium will vary depending on the particular factors of each case, including the subject's weight and the severity of the subject's condition. Nevertheless, the appropriate amount of agent can be readily determined by the skilled artisan.

In the method of the present invention, the agent which interacts with ZipA may be administered to a human or animal subject by known procedures, including, without limitation, oral administration and parenteral administration (e.g., by intradermal, intramuscular, intraperitoneal, intravenous, or subcutaneous injection). Preferably, the agent which interacts with ZipA is administered intraperitoneally. For oral administration, a formulation of the agent which interacts with ZipA may be presented as capsules, tablets, powders, granules, or as a suspension. The formulation may have conventional additives, such as lactose, mannitol, corn starch, or potato starch. The formulation also may be presented with binders, such as crystalline cellulose, cellulose derivatives, acacia, corn starch, or gelatins. Additionally, the formulation may be presented with disintegrators, such as corn starch, potato starch, or sodium carboxymethyl-cellulose. The formulation also may be presented with dibasic calcium phosphate anhydrous or sodium starch glycolate. Finally, the formulation may be presented with lubricants, such as talc or magnesium stearate.

For parenteral administration, the agent which interacts with ZipA may be combined with a sterile aqueous solution which is preferably isotonic with the blood of the subject. Such a formulation may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulations may be presented in unit or multi-dose containers, such as sealed ampules or vials. The formulations may be delivered by any mode of injection, including, without limitation, intradermal, intramuscular, intraperitoneal, intravenous, or subcutaneous.

It is also within the confines of the present invention that the agent which interacts with ZipA be administered to a subject infected with a bacterium, either alone or in combination with one or more antimicrobial drugs. Examples of antimicrobial drugs with which the agent may be combined include, without limitation, amphotericin B, fluconazole, flucytosine, itraconazole, and ketoconazole.

The present invention is described in the following example, which is set forth to aid in the understanding of the invention, and should not be construed to limit in any way the scope of the invention as defined in the claims which follow thereafter.

### EXAMPLE 1

1. Introduction

ZipA has been identified as an essential component of the cell-division machinery of Gram-negative bacteria, including E. coli, through its direct association with FtsZ (14). During the initial stage of cell division, FtsZ moves from the cytoplasm to the division site, where it assembles into the Z-ring. The resultant structure provides a platform from which to recruit other members of the Z-ring. Binding of ZipA to FtsZ occurs early in cell division, and independently of FtsA (15, 19). FtsA is required for the subsequent localization of additional components of the cell-division machinery, including FtsQ, FtsL, and FtsI (PBP3) (11, 29, 30). PBP3 directs the synthesis of new peptidoglycan as the septum is formed. Despite what is known about FtsA in organizing the cell division machinery, it is not known whether the localization of any proteins to the Z-ring is dependent on ZipA.

Cryptic prophages are resident in many E. coli strains. The cryptic prophage, Rac, is a 25-kb fragment located at min 29.5 in many E. coli K-12 strains (8), including MG1655, the strain sequenced by Blattner et al. (4). It encodes the Rac immunity region, as well as genes encoded by the early promoters—including recE, a gene that promotes recA-independent homologous recombination, and trgG, which functions in conjunction with the host gene trgH to transport potassium. Rac can be cured from the chromosome, resulting in strains that are sensitive to zygotic induction by parental strains (9). Zygotic induction of Rac is characterized by filamentation that results from expression of the kil gene. The filamentation phenotype is an indication that kil encodes an inhibitor of cell division. Prophage that carry mutations in the kil gene are no longer lethal to cured strains. Suppression of kil by overexpression of FtsZAQ, but not FtsAQ, indicates that the site of action is FtsZ.

The inventor sought to extend an understanding of ZipA by identifying proteins that interact with it. One caveat to such an approach is the observation that many genes involved in cell division are toxic when cloned on plasmids, occasionally even in the absence of a promoter sequence. Therefore, it seemed apparent that libraries passaged through E. coli would be underrepresented in these genes. In view of the extraordinary complexity of yeast two-hybrid libraries, it is difficult to use standard amplification techniques (e.g., the dispersal of single colonies on plates to allow independent growth) to amplify the libraries while maintaining their complexities. To avert this problem, the inventor used a recently developed two-hybrid library that attempts broader coverage of the E. coli genome through construction and amplification of the library exclusively in yeast. This library has been used to identify genes that interact with ZipA.

2. Materials and Methods

A. Strains, media, and plasmids

Yeast strains EGY48 and YM4271, bacterial strain KC8, and plasmids pLexA, pLexA(OP)8-lacZ, and pB42, were obtained from Clontech. E. coli strain TB1 was obtained from NEB. Strains SHy22 and SHy23 were generated by introducing pHO (kindly provided by Kim Arndt, Wyeth-Ayerst Research, Pearl River, N.Y.) into strain SHy9, and growing a transformant in 10 ml of SC-URA overnight. Cells were streaked onto a YPD plate, and allowed to grow for 3 days. This plate was then replica-printed onto an SC plate supplemented with 0.1% FOA. Colonies from this plate were patched onto a new YPD plate, grown overnight, and replica-printed onto an SPO plate. This plate was incubated at room temperature for 24 hours, then at 30° C. for 5 days. Patches producing asci then were incubated with Zymolyase. Spores were separated by the random-spores technique, then plated on YPD (17). SHy22 and SHy23 are strains from the same patch, and differ only by mating type. Strain SHy9, which was used to generate strains SHy22 and SHy23, was generated by growing strain CG1945 serially for two 10-ml overnight cultures, each with an inoculum containing about 10⁵ cells, then plating onto SC plates supplemented with 0.1% FOA.

Strain L40 was obtained from Invitrogen. Strain SHy19 was constructed by mating strain EGy48 with strain L40, and dissecting progeny. Diploids were sporulated, and asci were dissected using a microdissector; spores then were germinated on YPD plates. Colonies from germinated cells were scored for the phenotypes listed in Table 1. Yeast methods, including preparation of growth media, transformations, and tetrad analysis, were performed using standard protocols (1, 13). Other procedures are described below. NZCMG medium consists of 10 g of NZ amine, 5 g of NaCl, 1 g of CAA, 2 g of MgSO₄ heptahydrate, and 0.2% glucose per liter.

TABLE 1
Plasmids and yeast strains used in this example
Plasmids

| Lab Name | Common name/characterization | Source |
|---|---|---|
| SHp40 | pLexA | Clontech |
| SHp47 | pLexA-ZipA(23-284) | this example |
| SHp41 | pB42 | Clontech |
| SHp256 | pB42-FtsZ | this example |
| SHp42 | pLexA(OP)8-lacZ | Clontech |
| SHp63 | pLexA(Ade2) | reference #18 |
| SHp64 | pLexA(Ade2)-ZipA | this example |
| SHp198 | pLexA(Ade2)-FtsA | this example |
| SHp131 | pMalC2-lacZα | NEB |
| SHp132 | pMalC2-kil | this example |
| SHp156 | pMalC2-FtsZ | this example |
| SHp19 | pAS2-1 | Clontech |
| SHp102 | pAS2-1-FtsZ(311-383) | this example |
| SHp17 | pGAD424 | Clontech |
| SHp104 | pGAD424-ZipA(23-328) | this example |
| SHp296 | pGAD424-ZipAΔ1 | this example |
| SHp297 | pGAD424-ZipAΔ2 | this example |
| SHp298 | pGAD424-ZipAΔ3 | this example |
| SHp299 | pGAD424-ZipAΔ4 | this example |
| SHp112 | pDG148 | this example |
| SHp114 | pDG148-FtsZbs | this example |
| SHp113 | pDG148-kil | this example |

Yeast Strains

| Lab | Common | | |
|---|---|---|---|
| Name | Name | Genotype | Source |
| SHy5 | EGY48 | MATα ura3-52 his3 trp1 GAL1p-lexA(OP)6- | Clontech |
| | | LEU2 | |
| SHy6 | YM4271 | MATa ura3-52 his3-200 lys2-801 ade2-101 | Clontech |
| | | ade5::hisG trp1-901 leu2-3, 112 tyr1-501 | |
| | | gal4Δ gal80Δ | |
| SHy4 | CG1945 | Mata ura3-52 his3-200 ade2-101 lys2-801 | Clontech |
| | | trp1-901 leu2-3,112 gal4Δ gal80Δ | |
| | | LYS2::GAL1_{UAS}-HIS3_{TATA}-HIS3 | |
| | | URA3::GAL1_{UAS}-GAL1_{TATA}-lacZ | |
| SHy22 | | Matα ura3-52 his3-200 ade2-101 lys2-801 | this example |
| | | trp1-901 leu2-3,112 gal4Δ gal80Δ | |
| | | LYS2::GAL1_{UAS}-HIS3_{TATA}-HIS3 | |
| SHy23 | | Mata ura3-52 his3-200 ade2-101 lys2-801 | this example |
| | | trp1-901 leu2-3,112 gal4Δ gal80Δ | |
| | | LYS2::GAL1_{UAS}-HIS3_{TATA}-HIS3 | |
| SHy16 | L40 | MATa ura3-52 his3-200 ade2-101 | Invitrogen |
| | | ade5::hisG trp1-901 leu2-3,112 tyr1-501 | |
| | | LYS2::LexA(OP)_{UAS}-HIS3 URA3::LexZ(OP)_{UAS}- | |
| | | lacZ GAL⁺ | |
| SHy19 | | MATα ura3-52 his3 trp1 LYS2::LexA(OP)_{UAS}- | this example |
| | | HIS3 URA3::LexZ(OP)_{UAS}-lacZ | |

To construct plasmid SHp47, ZipA was amplified by PCR using oligo ZipA-5′, resulting in a PCR product that deleted the membrane-spanning portion of the ZipA gene product. This fragment was digested with Eco RI and Sal I, and ligated into pLexA to generate plasmid SHp47. Plasmid SHp256 was constructed by PCR amplification of the entire FtsZ gene using oligos that encoded Mfe I and Sal I sites, and ligation into Eco RI and Sal I sites of the pB42 vector.

Plasmid SHp64 was constructed by digesting plasmid SHp47 with Eco RI and Sal I, isolating the 0.9-kb fragment encoding ZipA, and ligating it into plasmid SHp63, which had been digested with identical enzymes. Plasmid SHp198 was constructed in essentially the identical manner, except that plasmid SHp100 was used as the source of FtsA. Plasmid pLexA(ADE2) (SHp63) was a generous gift from Kim Arndt (Wyeth-Ayerst Research, Pearl River, N.Y.). SHp132 was constructed by PCR amplification of the kil gene encoded by the construct recovered in the screen described below. kil was amplified using the following primers: 5′-GGCCGGCGGAATTCATGTTGCACATC ACTTCGGAACTG (SEQ. ID. NO. 3) and 5′-CCCGGGGTCGACTCATCACCATGAC TCCGCCTTTAC (SEQ. ID. NO. 4). The resulting 0.3-kb fragment was isolated, digested with Eco RI and Sal I, and cloned into pMal-C2×, which had been identically digested. The FtsZ insert used to construct SHp156 was obtained from plasmid SHp101 (pGAD424-FtsZ) after digesting with Eco RI and Sal I.

To construct plasmid SHp102, a segment of the FtsZ gene which encodes amino acid residues 311-383 was amplified by PCR using oligos that encoded an Eco RI site in frame with one in the pAS2-1 vector, and a Sal I site after the stop codon of FtsZ. To construct plasmid SHp104, a segment of the ZipA gene encoding amino acid residues 23-328 was amplified by PCR using oligos that encoded an Eco RI site in frame with one in the pGAD424 vector, and a Sal I site after the stop codon of ZipA.

Plasmids SHp296-299 were constructed by cloning PCR products of specific regions of ZipA into pGAD424, following digestion with Eco RI and Bam HI. Plasmid SHp296 used 5′-AAGAACGAGAATTCATGTTCCGCGAT (SEQ. ID. NO. 5) for the 5′-oligo. Plasmids SHp297 and SHp298 used 5′-AACCTGC TGAATTCATGGATAAACCG (SEQ. ID. NO. 6) for the 5′-oligo. Plasmid SHp299 used 5′-GAAGA TGAATTCGGCGTTGGTGAGGT (SEQ. ID. NO. 7) for the 5′-oligo. Plasmid SHp296 used 5′-GCTTCGGTGGATCCATTTATGGAGCAGGTT (SEQ. ID. NO. 8) for the 3′-oligo. Plasmid SHp297 used 5′-CATACGGCGGGGATCCTTAA AGCACGACACCG (SEQ. ID. NO. 9) for the 3′-oligo. Plasmids SHp298 and 299 used 5′-GACTGAGG ATCCTCAGGCGTTGGCGTCTTTGACT (SEQ. ID. NO. 10) for the 3′-oligo.

pDG148 is a B. subtilis/E. coli shuttle plasmid derived from pCED6 (36), pBR322, and pSI1 (37). pCED6 encodes the origin of replication in B. subtilis and a kanamycin resistance gene (36). pBR322 contributes the origin of replication in E. coli and the bla gene that encodes ampicillin resistance. pSI1 contributes the pSpac hybrid promoter and the E. coli lacI gene under the control of the B. subtilis penP gene (37). The pSpac promoter is a B. subtilis SPO1 promoter under the control of the lacI gene product (37). Plasmid SHp113 was constructed using 5′-CCCGGGGCTCTCAAGCTTAGGAGGATTT AGCATGGAGATAATTATGATTGC (SEQ. ID. NO. 11) and the 3′-oligo that was used to construct SHp132. These oligos were used to amplify the kil gene from plasmid SHp132. Plasmid SHp114 was constructed using 5′-CCCGGGCGTCTCA AGCTTAGGAGGATTTAGCATGTTGGAGTTGGAGTTCGAAACAAACATAGACGGC (SEQ. ID. NO. 12) and 5′-GCGCGCGCGGTCGACTTAGCCGCGTTTATTAC GGTTTCTT (SEQ. ID. NO. 13). These oligos were used to amplify the FtsZ gene from B. subtilis using pUC119-FtsZbs as a template. pUC119-FtsZbs was generously provided by David Keeney (Wyeth-Ayerst Research, Pearl River, N.Y.). Plasmids SHp113 and SHp114 were constructed by digesting the inserts with Bam BI and Sal I, and ligating into pDG148 that had been digested with HinD III and Sal I.

B. Cloning genes that interact with ZipA

A library was prepared from E. coli strain MG1655 genomic DNA that had been partially digested with Tsp509 I, Sau3A, Msp I, Mae II, or Taq(alpha) I. Genomic inserts were ligated into pB42 vector DNA that previously had been modified to include additional restriction sites for cloning fusions to the B42 activation domain. This plasmid DNA was digested with Eco RI, BamH I or Cla I, and the appropriate genomic DNA was mixed and ligated. The library was transformed directly into yeast strain YM4271 by lithium acetate-mediated transformation.

For screening, 200 ml of SC-His-Ura were inoculated with a 5-ml overnight culture of EGY28, transformed with SHp47 and SHp42, and grown to a density of 0.8 OD. This culture was pelleted, and resuspended in 20 ml of YPD. Frozen aliquots of the library contained in the YM4271 host were revived by thawing on ice, then incubated in 10 ml of YPD for 2 hours at 30° C. with mild agitation. Titering data indicated that 1.1×10⁷ His⁺Ura⁺Trp⁺ diploids were plated. The diploids were plated onto 20 large SD-LUTH/GalRaf plates (12), and incubated for 5 days at 30° C. Approximately 10⁵ colonies grew at this stage, so the plates were replica printed onto SD-LUTH/GalRaf/X-Gal plates (12). 124 colonies were recovered that were Leu⁺LacZ⁺.

C. Rescue and DNA sequencing of clones

All diploids containing prey plasmids that putatively interacted with ZipA were inoculated into 5 ml of −Trp medium and grown overnight. The cultures were pelleted and resuspended in 250 μl of solution P1 from the Qiagen Turbo Miniprep kit that had been supplemented with 500 μg/ml Zymolyase (ICN Biologicals). The cultures then were incubated at 37° C. for 30 min. Further steps in the purification of plasmid DNA were performed as per the instructions of the manufacturer. 3 μl of the eluted DNA miniprep was used to transform E. coli strain KC8 by electroporation.

D. Two-hybrid assays

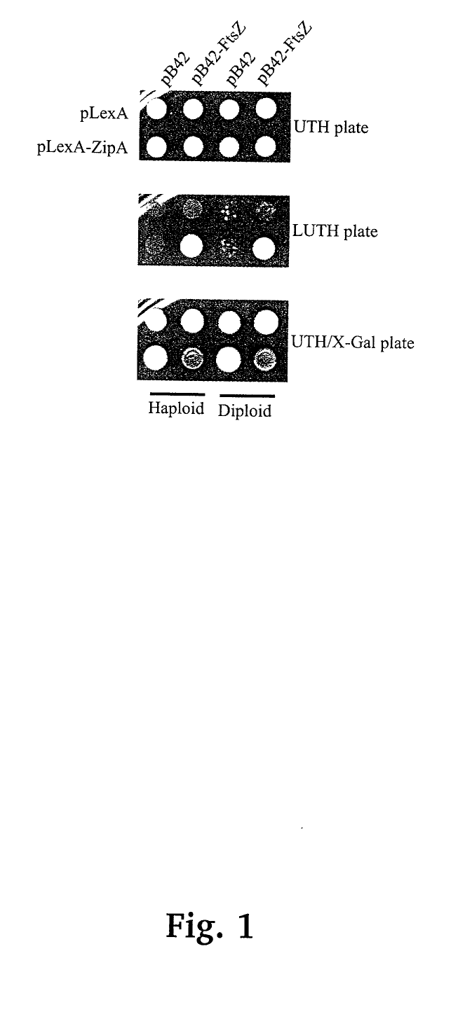

Testing of ZipA as an appropriate bait for a two-hybrid screen was determined by assaying its interaction with FtsZ in both haploids and diploids. In haploids (the type of strain used for most two-hybrid screens and tests), plasmids were transformed into strain SHy5 (EGY48) by lithium acetate transformation, as indicated in FIG. 1. For the diploid tests (the strain background that results from the mating-based screening), baits to be tested were transformed into SHy5 along with SHp42 (plexA(OP)8-lacZ), with selection for His⁺Ura⁺ transformants, and prey plasmids were transformed into strain SHy6 (YM4271), with selection for Trp⁺ transformants. Strains were streaked into grids on selective plates, and allowed to grow overnight. The streaks then were replica plated orthogonally onto a YPD plate, so that all baits would mate with all preys (12). Strains were allowed to grow overnight, and then were replica printed onto a plate that selected for His⁺Trp⁺ diploids, which grew at the intersections of the crossed streaks. These diploids were either tested by replica printing the patches onto plates to test for leucine prototrophy, or grown as 5 ml overnight cultures and spotted onto plates for testing. Plates for testing are indicated in FIG. 1. Analysis of clones was performed as described above, except that the bait plasmids were transformed into strain SHy108, selection for Ade⁺ transformants replaced selection by His⁺, and assaying for histidine prototrophy replaced assaying for leucine prototrophy.

E. β-Galactosidase assays

Overnight cultures (5 ml) of yeast strains to be assayed for LacZ activity were aliquoted into a 96-well microtiter plate (100μl per well). Samples were mixed with 100 μl of lysis buffer and substrate (40 μl of Promega Cell Lysis Buffer, 40 μl of 0.125 mg/ml MUG (Sigma), and 20 μl of 10×β-galactosidase assay salts (26)). Samples were incubated at 30° C. for 4-8 hours with shaking, then read on a Victor II fluorescence plate reader from Wallac. Fluorescence intensity increased with time. After 8 hours, negative-control wells showed about 400 units, whereas positive control wells showed about 50,000 units. Data presented here are the averages of quadruplicate measurements, and are representative of three independent assays.

F. Phase-contrast microscopy

Samples for phase-contrast microscopy were grown in 2×YT medium until the cultures had an OD of 0.6. Samples were induced with 1 mM IPTG (final conc.), and grown for an additional 4 hours. 1 ml of each culture was pelleted and washed with water. The cultures then were resuspended in 50 μl of water. 50 μl of 2% glutaraldehyde was added to the cultures, and the samples were incubated for 30 min at room temperature. The samples were washed again, then spread onto glass slides that had been coated with poly-L-lysine and covered with glass coverslips. Slides were viewed with a Zeiss Axioplanin microscope.

G. In vitro interaction assay

E. coli ZipA protein (a generous gift of Elizabeth Glasfeld, Wyeth-Ayerst Research, Pearl River, N.Y.) was purified as previously described (16). Maltose-binding protein fusions, containing the C-terminus of FtsZ, full length OrfE, or the LacZa fragment, were purified using amylose resin, as described by the manufacturer (NEB). Resin bound with the fusion proteins were quantified by spectrophotometric analysis of the eluted proteins, and by examination of proteins bound to the resins after PAGE and staining with Coomassie Blue. For the interaction assay, 10 μg of fusion protein, bound to 20 μl of amylose resin, were incubated with 20 μg of ZipA for 1 hour at room temperature, in a total volume of 200 μl of TBS. The resin was pelleted in a microfuge, and washed two times with 200 μl of TBS. The samples were resuspended in 200 μl of 20 mM maltose in TBS, and separated on a 10% PAGE gel. 20 μl then were examined by Western blot using anti-ZipA antibodies that previously have been characterized.

H. Fluorescence microscopy

GFP-FtsZ was examined by fluorescence microscopy, as previously described (15, 16).

3. Results

A. Identification of genes that interact with ZipA

ZipA is an essential component of the E. coli cell division machinery. However, to date, all that is known about ZipA concerns its interaction with FtsZ (14-16, 19, 21, 22). While ZipA's interaction with FtsZ is a major part of its role in cell division, it is not clear whether this is its only role, nor whether it accomplishes this role with additional proteins. To gain some insight into these issues, the inventor used a new yeast two-hybrid (Y2H) library to identify additional genes that encode proteins which interact with ZipA. This library differs from other E. coli genomic libraries because it was constructed without passage through E. coli at any step. The logic behind this modification is based on the observations that many E. coli genes are toxic when expressed on plasmids, and that the passage and amplification steps of a library construction will cause a severe underrepresentation of such plasmids. The inventor has shown that such underrepresentation of toxic genes can be limited by forgoing the use of E. coli as a host, and by keeping the expression of such clones repressed in yeast as well. Use of the present library resembles use of other libraries of pB42, except that baits and reporters must be introduced by mating rather than through transformation with the library DNA (3, 12).

In the first stage of this screen, the inventor had to determine if ZipA was a sufficiently robust target for a mating-based two-hybrid screen strategy. The ZipA gene was cloned into pLexA, and its interactions with FtsZ were characterized in both the haploid yeast strain EGY48 and the diploid EGY48×YM4271 backgrounds. For the interactions to be tested, the haploid strains were constructed by cotransformation, while the diploid strains were constructed by transforming haploid strains with either the baits and a lacZ-based reporter plasmid (for EGY48) or preys (for YM4271). Transformants then were mated in rows and columns to form the diploids, providing a definitive test for the appropriateness of the target for the mating-based strategy of this library.

pLexA and pLex-ZipA were compared with pB42 and pB42-FtsZ. The results are set forth in FIG. 1. In the top panel, haploid and diploid cells containing the indicated plasmids are spotted onto a UTH plate, which ensures that the cultures are viable for plating. The plates in the remaining panels are designed to determine whether a specific interaction between ZipA and FtsZ can be identified in this system. In the second panel, activation of the lexA(OP)₆-LEU2 reporter is examined. As can be seen, only the strains that express the lexA-ZipA and B42-FtsZ fusions will grow on this plate. In the third panel, expression of the lexA(OP)₈-lacZ reporter is depicted. This plate tests for the expression of the lacZ reporter, without also testing for the activity of the LEU2 reporter. Only the strains that express the ZipA and FtsZ fusions show indicator activity. In each of these tests, the haploid results are identical to the diploid results, with the exception that the diploid strains spotted onto the LUTH/GalRaf plate (middle panel) have a higher background than the haploid strain. However, this background clearly can be distinguished from the robust growth of the strain expressing the specific interaction. From this examination, it was determined that ZipA was a target that could be used in the mating-based screen design.

The screen was initiated by reviving the library in YPD, and mixing this with a culture of EGY48pLexA-ZipA. The culture was allowed to mix for 6 hours, to permit zygotes to form, and then the culture was plated on LUTH/GalRaf plates. After 2 days, a high background was observed, where about 10% of the Ura⁺Trp⁺His⁺ colonies were growing on the selective plate. This potential problem was indicated in FIG. 1, and has been described previously (12). As a result, the plates were replica printed onto LUTH/GalRaf/X-Gal plates. After 5 days, blue colonies were rescued, and 124 clones were recovered. Plasmid DNA was obtained and used to transform E. coli KC8. These transformants contained any of the three plasmids expressed in yeast (the bait, the prey, or the reporter). Prey plasmids were selected by taking advantage of the fact that the TRP1 locus in the prey plasmid can complement the trpB mutation in strain KC8. Transformants were plated on M9 minimal plates supplemented with leucine and uracil. This selected for only those cells that were transformed with the prey plasmid. When transformants for each rescued clone were spotted as a pool in an array, and allowed to grow overnight, some growth inhibition was observed.

For transformants that were stable in E. coli, new transformations in yeast were made from DNA purified from single colonies. These transformants were assayed for the specificity of their interaction with ZipA by creating new diploids against SHy52 (EGY48pLexA) and SHy53 (EGY48pLexA-ZipA). Prey plasmids that interacted with both strains, as determined by induction of the lacZ reporter in a plate assay, were discarded as either activating transcription independently of the bait plasmid, or by interacting with LexA. For prey plasmids that did not appear to be stable in E. coli, the bait plasmid was lost by segregation through growth in non-selective medium (SD-Ura-Trp); bait and control plasmids then were retransformed into the diploid.

Prey plasmids that appeared specific for the pLexA-ZipA-expressing strain were analyzed by sequencing their inserts. From the two classes of prey, a list of putative interactors was compiled. The genes listed were searched against the literature to determine what was known about them. In general, numerous membrane proteins were identified, including many uncharacterized transporters. It was not apparent to the inventor why these would be identified; therefore, they were held aside for later determination as to whether the interactions represent biological functions, or whether an uncharacterized physical property of the LexA-ZipA fusion facilitates a nonspecific interaction with membrane proteins. The fusion to ZipA began after the membrane spanning region, so the inventor was not identifying proteins that interacted with that segment of ZipA. Fusions to the FtsZ gene were isolated four times.

One gene identified in this screen previously had been shown to confer a cell division defect, but its early characterization suggested an interaction with FtsZ rather than ZipA (8). This gene, the kil locus of the cryptic prophage Rac, expresses a small protein identified as OrfE. This clone exhibited slow, unstable growth when retransformed into E. coli, and the insert was characterized by PCR rather than by plasmid prep.

B. kil interacts with ZipA, but not FtsA or FtsZ, in the yeast two-hybrid assay

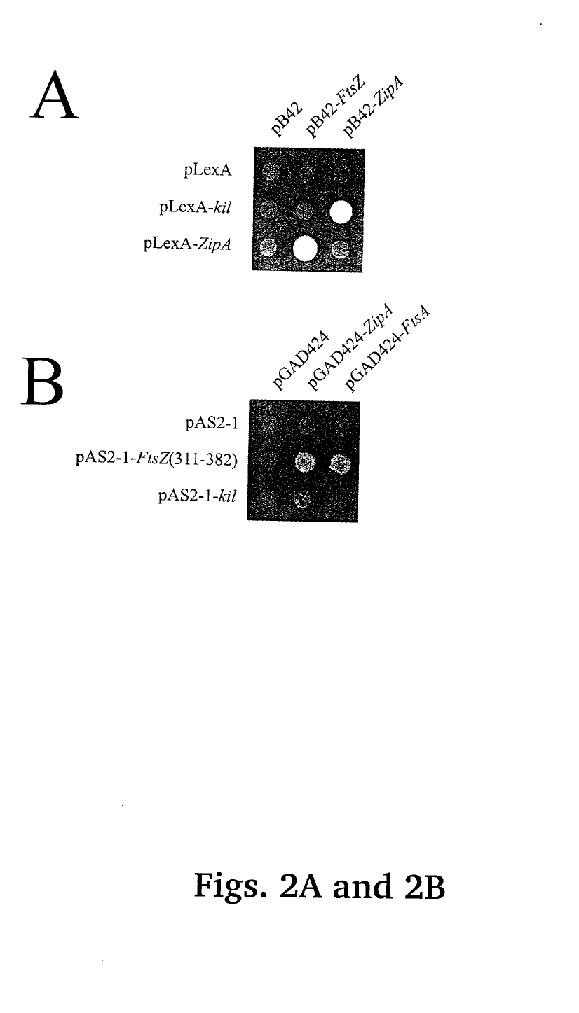

The ability of kil to interact with ZipA, FtsZ, and FtsA was examined extensively in the two-hybrid system. The assay included examination of kil as a fusion to the DNA-binding and the transcription-activation proteins of both the Gal and Lex Y2H systems. As can be seen in FIG. 2, kil shows a specific interaction for ZipA in the Y2H system. In FIG. 2A, kil is examined as a fusion to the LexA DNA-binding protein. In this example, the genes tested as LexA fusions were transformed into strain SHy19, a segregant of a diploid that results from crossing SHy5 (EGY48) with SHy16 (L40). This strain is MATα, which allows mating to SHy6, and which carries the GAL1-HIS3 and GAL1-LacZ reporters, for testing putative interactions. The marker for the pLexA plasmid was changed to ADE2 from HIS3 (to allow use of the HIS3 reporter). Fusions to the activation domain in plasmid pB42 were transformed into strain SHy6. Transformants were mated, and the resulting diploids were assayed on plates without histidine. The data show that the kil gene product, OrfE, interacts with ZipA, but not with FtsZ. In contrast, the LexA-ZipA fusion interacts with the B42-FtsZ fusion, but not with ZipA, in this assay, suggesting that the FtsZ gene product is functional in this assay.

In FIG. 2B, the interaction of kil with ZipA and FtsA is examined in the Gal system. Diploid strains were constructed in a manner similar to that described above. The interaction of the Gal4_{bd}-Kil fusion with the Gal4_{ad}-ZipA and the Gal4_{ad}-FtsA fusions was compared to a fusion of the Gal4_{bd} with the last 72 residues of FtsZ. The FtsZ(311-383) fragment previously had been shown to interact specifically with ZipA and FtsA in yeast. It contains the conserved and well-characterized binding site for ZipA and FtsA. This segment of the FtsZ gene was used because larger segments fused to Gal4_{bd} result in an unacceptably high level of auto-activation. In the present example, the Gal4_{bd}-FtsZ(311-383) fusion interacted with fusions to both ZipA and FtsA, as shown previously, whereas the Gal4_{bd}-OrfE fusion interacted only with ZipA. The interaction in this example was weaker than the interaction of FtsZ(311-383) with either ZipA or FtsA. This suggests that kil is not expressed as well in the Y2H system. Nevertheless, the level of interaction between kil and ZipA is sufficiently strong, relative to the vector control, to allow a conclusion that kil is interacting in this system. No such interaction was seen for the fusion with FtsA.

C. kil produces a lethal cell-division phenotype when expressed as a maltose-binding protein (MBP) fusion

In order to characterize the interaction of OrfE with ZipA more thoroughly, and, in particular, to show that this interaction is direct, the inventor sought to express and purify OrfE as a fusion protein for in vitro studies. OrfE was fused to the maltose-binding protein (MBP), as was the FtsZ(311-383) segment which was used as a positive control. The pMalC-2×vector was used as a negative control. This plasmid encodes the LacZα fragment that was fused to the MBP. Plasmids were transformed into E. coli strain TB1 for expression of the fusion proteins. Transformed strains showed marked differences in their growth characteristics and morphology. For example, strains expressing fusions to FtsZ(311-383) or OrfE did not grow as well as the strain expressing the MBP-LacZα fusion, even in the absence of IPTG. Such differences were attributed to the different fusions expressed in each strain.

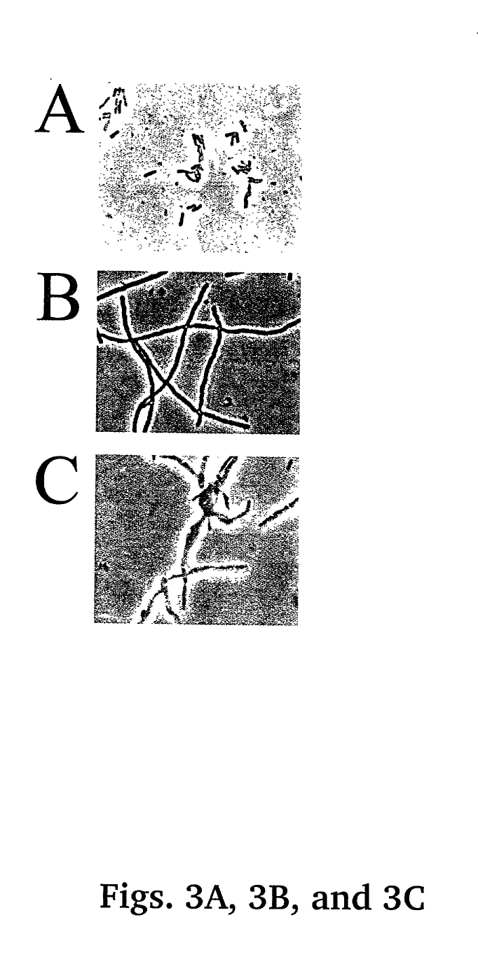

Examples of the morphology of strains exposed to 1 mM IPTG for 4 hours are illustrated in FIG. 3. The top panel shows a strain expressing the pMalC-2× vector. The middle and lower panels show strains carrying the pMalC-2-FtsZ(311-383) and the pMalC-2-kil plasmids, respectively. Expression of the FtsZ and OrfE fusions shows a strong cell-division phenotype, including a high degree of filamentation. In the lower panel, additional observations can be made, including a loss of cell shape and severe bending of cells—phenotypes previously observed in a characterization of kil/OrfE (8). In the present example, the reproduction of these previously observed phenotypes indicates that cell-division genes expressed as fusion proteins retain their biological activity.

D. OrfE interacts with ZipA in vitro

The constructs described in the previous section were used to express fusion proteins that could be purified and characterized in vitro. The three fusion proteins—MBP-LacZα, MBP-FtsZ(311-383), and MBP-OrfE—were all bound to amylose beads and washed thoroughly. Wild type ZipA was purified independently. PAGE analysis and Coomassie staining showed that all proteins were free of any contaminants approaching stoichiometric levels, and no protein co-purified specifically with any of the proteins fused to MBP.

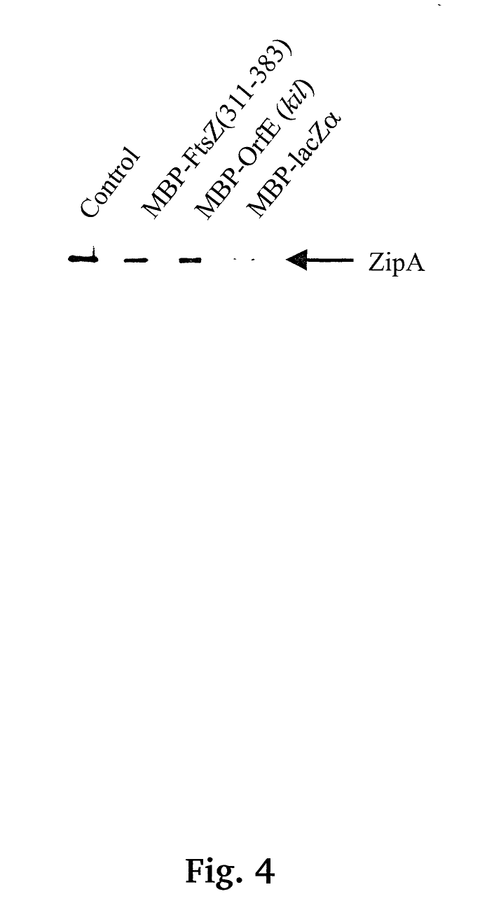

Beads used to purify the fusion proteins were tested for their ability to precipitate purified ZipA in vitro. MBP-fusion proteins, bound to amylose resin, were used to precipitate purified ZipA. Beads were incubated with ZipA, and washed. A sample then was run on a PAGE gel, transferred to nitrocellulose, and probed with a polyclonal anti-ZipA antibody. The result is seen in FIG. 4. At the left of the figure is a sample of the purified ZipA, which was loaded for comparison. For the three samples tested in the precipitation assay, the first (lane 2) shows the ZipA protein precipitated by the MBP-FtsZ(311-383) fusion. Lane 3 shows ZipA after precipitation with MBP-OrfE. Lane 4 shows ZipA precipitated by MBP-lacZα. As FIG. 4 shows, ZipA binds strongly to the C-terminus of FtsZ and to OrfE, as compared with the LacZα control.

The interaction with OrfE appears to modify ZipA in vitro. In the other conditions examined in this assay, ZipA appeared as a single band of the same Mᵣ. In contrast, the ZipA that had been precipitated by OrfE appeared as a doublet, suggesting that it had separated into two forms. Since this assay was run without ATP or any other agent typically used to modify proteins, it may be that some portion of ZipA had undergone a structural change as a result of its interaction with OrfE. The nature of this putative change is unclear, but observations made during the purification of ZipA indicated that it existed in more than one form, and that these forms may represent structural heterogeneity.

E. OrfE interacts with the carboxyl (ZBD) region of ZipA

ZipA is comprised of several domains or regions. A transmembrane region at the amino terminus is followed by a charged region, a P/Q rich region, and a C-terminus domain of about 150 residues. ZipA homologues from other bacteria show sequence similarity along all of these segments, but the homology is particularly striking in the C-terminus domain. Deletion studies have shown that this region is also required for ZipA binding to FtsZ. Deletions through the first 185 residues do not affect the interaction with FtsZ, but deletions of the last 20 residues—the FtsZ-binding domain, or ZBD (16)—result in a loss of binding to FtsZ.

The inventor sought to characterize the binding of OrfE to ZipA as a means of producing a preliminary model for how OrfE may function to inhibit cell division. Deletions of ZipA were constructed as fusions to the Gal4_{ad}, and their interactions were analyzed in a two-hybrid assay. The results set forth in Table 2 show that the interaction was not observed when only the less-conserved P/Q region of ZipA was tested in the ZipA(35-185) domain.

TABLE 2
Interaction of FtsZ and kil with ZipA deletions in
the two-hybrid system

| | pGAD424 fusion | | | | | |
|---|---|---|---|---|---|---|
| | | ZipA | ZipA | ZipA | ZipA | ZipA |
| | none | (35- | (35- | (185- | (185- | (62- |
| pAS2-1 fusion | (vector) | 284) | 185) | 302) | 328) | 328) |
| none (vector) | 621.25 | 831.75 | 930.5 | 1123.75 | 877.25 | 743.25 |
| FtsZ(311-383) | 997 | 8621.5 | 971.25 | 828.25 | 2632.75 | 7429 |
| OrfE | 1229.5 | 7581.25 | 1136.25 | 720.75 | 1910 | 8527 |

Similarly, deletion of the C-terminus, shown previously to include critical residues for ZBD function (16), also abrogated the interaction with both fusions tested. The interaction with the minimal ZBD showed a reduced interaction with both fusions, indicating that some structural or steric factors affected this fusion. However, an interaction with both bait fusions was still seen, supporting the role of the ZBD in mediating interactions with both FtsZ and OrfE. kil was cloned with a fusion of ZipA that was missing the membrane-spanning domain (residues 1-34), so this segment was not tested further.

F. Inhibition of cell division by expression of kil affects the structure of the Z-ring

In the last few years, methods for examining E. coli cells by fluorescence methods have improved significantly. For this reason, the inventor wanted to examine the effects on the cell-division machinery that could be observed following expression of kil. kil was introduced into E. coli under the control of the pBAD promoter in a strain carrying a pLac::GFP-FtsZ lysogen. In the absence of arabinose, but with 50 μM IPTG, cells grew normally, and showed normal Z-rings. Upon addition of arabinose, cells could be seen to filament. Fewer Z-rings could be observed in filamenting cells, and the extent of the phenotype was more significant as the concentration of arabinose was increased.

G. Expression of kil inhibits cell division in Bacillus subtilis

The inventor also investigated the role of OrfE in regulating cell division in B. subtilis. Expression of kil in this Gram-positive organism permitted examination of the conservation of cell-division machinery in two distinct microorganisms: E. coli and B. subtilis. All prokaryotes except Chlamidia have at least one FtsZ gene, and these genes generally encode the conserved C-terminus region that is the primary determinant in binding and localizing FtsA and ZipA. FtsA itself, though, is found in only a subset of these bacteria, and ZipA has not been found in any Gram-positive organism. Expression of kil would not be expected to produce a phenotype in B. subtilis, if it functions by targeting ZipA. Inhibition of cell division by expression of kil would indicate one of the following: (a) that a paralogue of ZipA, existing in B. subtilis, is capable of being targeted by OrfE, or (b) that OrfE targets a second function in B. subtilis that may or may not be conserved in E. coli.

The inventor expressed kil under the moderately regulated pSpac promoter. As a positive control, the B. subtilis FtsZ gene was also expressed. Plasmids containing both genes were introduced into B. subtilis strain 168 by transformation, and cultures for expression studies were prepared. During this phase of the experiment, it was clear that expression of kil in B. subtilis produced strong phenotypes. The transformation efficiency of the plasmids expressing the FtsZ and kil genes was drastically reduced to about 5-10% of a vector control. Colonies that did arise were not visible for 2 days after transformation, and were smaller than colonies that grew from the control transformation. Overnight cultures were less dense than the strains transformed with the vector control. This would be expected from any gene that is toxic to B. subtilis because, although the pSpac promoter is regulated by IPTG, it has a high basal activity.

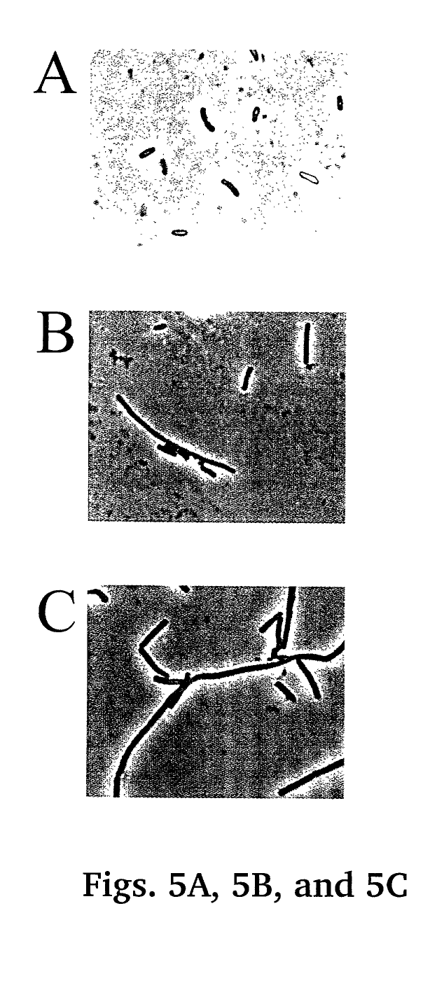

In order to ascertain whether expression of kil confers a defect in cell division, the inventor examined cells by phase-contrast microscopy. Given the strong effects seen in the transformation and growth of the plasmids expressing FtsZ and kil cells were examined after 4 hours of growth without induction by IPTG. The results are set forth in FIG. 5. Cells transformed with the vector control are shown in FIG. 5A. The cells were normal in appearance. In FIG. 5B, expression of FtsZ shows a cell-division defect, as characterized previously. The cells were elongated and growth was inhibited, as described above. In FIG. 5C, the effect of expression of kit in B. subtilis is shown. As was seen for E. coli, the cells were elongated and misshapen. The loss of integrity to the cell wall of B. subtilis was less severe than that for E. coli in FIG. 3. However, cells were twisted and bent, indicating a weak cell wall.

4. Discussion

ZipA is a relatively new component of the cell-division machinery of Gram-negative bacteria (14). It appears neither to be dependent on, nor required for, the recruitment of any protein in the cell-division machinery, other than the establishment of the Z-ring (15, 19, 20). It is for this reason that the inventor sought to characterize the role of ZipA in cell division, by identifying other proteins with which it may interact.

In this example, the inventor has described ZipA as the target of OrfE, the product of the cryptic prophage Rac gene kil (8). kil is a name commonly given to genes that interfere with cell growth. Typically, the interference is associated with the inhibition of cell division and cell lysis, particularly for kil genes found in lambdoid phages and in phage Mu. kil genes function in promoting cell lysis as the phage takes over the host. The kil genes of phages lambda, H19B, and HKO-22 (23, 24) share significant homology, and some elements are conserved in the phage Mu kit gene (28). The kil gene of phage P4 results in strong inhibition of cell division, but it is a nested gene within the longer eta gene (10). The Rac kil gene appears to be distinct from these other phage genes. All of these kil genes, however, are distinct from the kil loci of plasmid incompatibility systems (2).

Other small proteins also regulate cell division in E. coli, e.g., the host protein MinE, and gene products of other cryptic phages, such as the Qin gene dicB (6, 25, 32). These proteins are not clearly homologous, and do not have a common mechanism of action. In particular, MinE is not involved in lysis; rather, it regulates placement of the Z-ring through its interactions on the MinCD complex (25, 26, 31). These regulators are all very small, ranging from 60 to 100 residues in length. Further complicating the nomenclature/function issue with regard to OrfE and cell division, a B. subtilis gene, maf, has been characterized as a homologue of the E. coli gene ore, and has been shown to have a role in septum formation (5). Both genes are located in or near the mreBCD loci of the bacterium, and encode 21-kDa proteins which may be associated with PBP3.

Currently, it is not clear whether inhibition of FtsZ-ZipA interaction is the precise mechanism of action of OrfE. Two results from the present example illustrate this point. First, the interaction of OrfE with ZipA in vitro appeared more complex than the interaction of OrfE with the C-terminus of FtsZ. In the former, a ZipA doublet was seen in Western blots, suggesting that a change in the ZipA structure had occurred; however, in the latter, binding to the FtsZ C-terminus appeared to leave ZipA unchanged. Second, and more importantly, the inhibition of cell division by expression of OrfE apparently occurred through more than simply a depletion of ZipA. Depletion of ZipA caused filamentation and death, but expression of OrfE also caused additional catastrophic changes to the cell wall. These changes included a loss of cell wall integrity and a loss of shape. This indicates either that the OrfE-ZipA complex produces a dominant effect on cell wall remodeling that occurs during growth and division, or that uncomplexed OrfE has an additional target besides ZipA. The loss of the Z-ring in strains expressing kil was similar to, but more severe than, the effect of depletion of ZipA on the Z-ring. It is possible that OrfE completely inhibits ZipA much faster than what was seen in ZipA depletion experiments. In such a case, the function of OrfE could be specific for ZipA, but the phenotypes of these two experimental systems (expression of kil versus depletion of ZipA) could result from different kinetics of ZipA inhibition.

### Cited References

(1) Adams et al., Methods in Yeast Genetics. (Cold Spring Harbor: Cold Spring Harbor Press, 1997).

(2) Altieri et al., Expression of the cloned ColE1 kil gene in normal and Kil^{r} E. coli. J. Bacteriol., 168:648-54, 1986.

(3) Bendixen et al., A yeast mating-selection scheme for detection of protein-protein interactions. Nucleic Acids Research, 22(9) :1778-79, 1994.

(4) Blattner et al., The complete genome sequence of Escherichia coli K-12. Science, 277:1453-62, 1997.

(5) Butler et al., Amplification of the Bacillus subtilis maf gene results in arrested septum formation. J. Bacteriol., 175(10):3139-45, 1993.

(6) Cam et al., Identification and sequence of gene dicB: translation of the division inhibitor from an in-phase internal start. Nucleic Acids Research, 16(14):6327-38, 1988.

(7) Campbell, A., Cryptic Prophage. In Escherichia coli and Salmonella typhimurium, vol.2., F. C. Neidhardt (ed.) (Washington, D.C.: ASM Press, 1996) 204-46.

(8) Conter et al., Identification of a new inhibitor of essential division gene ftsZ as the kil gene of defective prophage Rac. J. Bacteriol., 178:5100-04, 1996.

(9) Feinstein and Low, Zygotic induction of the rac locus can cause cell death in E. coli. Molecular and General Genetics, 187:231-35, 1982.

(10) Forti et al., Translation of two nested genes in bacteriophage P4 controls immunity-specific transcription termination. J. Bacteriol., 181(17):5225-33, 1999.

(11) Ghigo et al., Localization of FtsL to the Escherichia coli septal ring. Molecular Microbiology, 31(2):725-37, 1999.

(12) Golemis and Brent, Searching for interacting proteins with the two-hybrid system III. In The Yeast Two-Hybrid System, P. A. Bartel and S. Fields (eds.) (Oxford, UK: Oxford University Press, 1997).

(13) Gutherie and Fink, Guide to Yeast Genetics and Molecular Biology. In Methods of Enzymology, vol. 194 (San Diego: Academic Press, 1991).

(14) Hale and De Boer, Direct binding of FtsZ to ZipA, an essential component of the septal ring structure that mediates cell division in E. coli. Cell, 88:176-85, 1997.

(15) Hale and De Boer, Recruitment of ZipA to the septal ring of Escherichia coli is dependent on FtsZ and independent of FtsA. J. Bacteriol., 181(1):167-76, 1999.

(16) Hale et al., ZipA-induced bundling of FtsZ polymers mediated by an interaction between C-terminal domains. J. Bacteriol., 182(18):5153-66, 2000.

(17) Rockmill et al., Spore Enrichment. Methods Enzymol., 194:146-49, 1991.

(18) Herskowitz and Jensen, Putting the HO gene to work: practical uses for mating-type switching. Methods Enzymol., 194:132-46, 1991.

(19) Liu et al., Recruitment of ZipA to the division site by interaction with FtsZ. Molecular Microbiology, 31(6):1853-61, 1999.

(20) Ma et al., Interactions between heterologous FtsA and FtsZ proteins at the FtsZ ring. J. Bacteriol., 179(21):6788-97, 1997.

(21) Mosyak et al., The bacterial cell-division protein ZipA and its interaction with the FtsZ fragment revealed by X-ray crystallography. EMBO J., 19(13):3179-91, 2000.

(22) Moy et al., Solution structure of ZipA, a crucial component of Escherichia coli cell division. Biochemistry, 39(31):9146-56, 2000.

(23) Neeley and Friedman, Arrangement and functional identification of genes in the regulatory region of lambdoid phage H-19B, a carrier of a Shiga-like toxin. Gene, 223(1-2):105-13, 1998.

(24) Oberto et al., Structure and function of the nun gene and the immunity region of the lambdoid phage HK022. J. Mol. Biol., 207(4):675-93, 1989.

(25) Pichoff et al., Deletion analysis of gene minE which encodes the topological specificity factor of cell division in Escherichia coli. Molecular Microbiology, 18(2):321-29, 1995.

(26) Raskin and de Boer, The MinE ring: An FtsZ-independent cell structure required for selection of the correct division site in E. coli. Cell, 91:685-94, 1997.

(27) Redfield and Campbell, Structures of cryptic prophage. J. Mol. Biol., 198:393-404, 1987.

(28) Waggoner et al., Identification of the bacteriophage Mu kil gene. Virology, 173:378-89, 1989.

(29) Wang et al., FtsI and FtsW are localized to the septum in Escherichia coli. J. Bacteriol., 180(11):2810-16, 1998.

(30) Weiss et al., Localization of FtsI (PBP3) to the septal ring requires its membrane anchor, the Z ring, FtsA, FtsQ, and FtsL. J. Bacteriol., 181(2):508-20, 1999.

(31) Zhang et al., The relationship between hetero-oligomer formation and function of the topological specificity domain of the Escherichia coli MinE protein. Molecular Microbiology, 30 (2) :265-73, 1998.

(32) Zhao et al., Proper placement of the Escherichia coli division site requires two functions that are associated with different domains of the MinE protein. Proc. Nat. Acad. Sci., 92:4313-17, 1995.

(33) Modern Techniques of Peptide and Amino Acid Analysis (New York: John Wiley & Sons, 1981).

(34) Bodansky, M., Principles of Peptide Synthesis (New York: Springer-Verlag New York, Inc., 1984).

(35) Beers and Berkow, eds., The Merck Manual of Diagnosis and Therapy, 17^{th} ed. (Whitehouse Station, N.J.: Merck Research Laboratories, 1999) 284-86, 924-25, 1088-92, 1101-03, 1156, 1159-60, 1431-32, 1884, 2172, 2175.

(36) Donnelly and Sonenshein, Promoter-probe plasmid for Bacillus subtilis. J. Bacteriol., 157:965-67, 1984.

(37) Yansura and Henner, Use of the Escherichia coli lac repressor and operator to control gene expression in Bacillus subtilis. Proc. Nat. Acad. Sci., 81:439-43, 1984.

All publications mentioned herein above are hereby incorporated in their entireties. While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. A method for identifying an agent which interacts with ZipA, comprising the steps of:
(a) contacting a candidate agent with ZipA, in the presence of OrfE; and
(b) assessing the ability of the candidate agent to inhibit OrfE-ZipA interaction.

2. The method of claim 1, further comprising the steps of:
(c) contacting the candidate agent with a bacterium; and
(d) determining if the agent has an effect on proliferation of the bacterium.

3. The agent identified by the method of claim 1.

4. A complex comprising OrfE and ZipA.

5. The complex of claim 4, wherein ZipA comprises the C-terminus domain (residues 176-328) of ZipA.

6. A method for inhibiting proliferation of a bacterium, comprising contacting the bacterium with an amount of the agent of claim 3 effective to inhibit proliferation of the bacterium.

7. The method of claim 6, wherein the bacterium contains ZipA.

8. The method of claim 7, wherein the bacterium containing ZipA is Escherichia coli.

9. A method for treating bacterial infection in a subject infected with a bacterium, comprising administering to the subject an amount of the agent of claim 3 effective to treat the bacterial infection in the subject.

10. The method of claim 9, wherein the bacterium contains ZipA.

11. The method of claim 10, wherein the bacterium containing ZipA is Escherichia coli.

12. The method of claim 9, wherein the agent is administered orally, intradermally, intramuscularly, intraperitoneally, intravenously, or subcutaneously.
